(19) United States
(12) Patent Application Publication
(10) Pub. No.: US 2009/0208408 A1
(43) Pub. Date: Aug. 20, 2009

(54) USE OF VCAM-1 LIGANDS FOR DETECTING AND/OR TREATING CARDIOVASCULAR DISEASES

(75) Inventors: Didier Boturyn, La Buisse (FR); Laurent Riou, Grenoble (FR); Catherine Ghezzi, Grenoble (FR); Daniel Fagret, Grenoble (FR); Pascal Dumy, Allevard (FR)

Correspondence Address: STITES & HARBISON PLLC, 1199 NORTH FAIRFAX STREET, SUITE 900, ALEXANDRIA, VA, 22314, US

(73) Assignee: UNIVERSITE JOSEPH FOURIER, Grenoble Cedex (FR)

(21) Appl. No.: 11/576,480

(22) PCT Filed: Sep. 30, 2005
(86) PCT No.: PCT/FR2005/002423

§ 371 Date: Jan. 12, 2009

(30) Foreign Application Priority Data
Oct. 1, 2004 (FR) 0410420

(51) Int. Cl. A61K 51/00; A61K 49/00; A61K 38/00

(52) U.S. Cl. 424/1.11; 424/9.1; 424/9.3; 424/9.6; 514/12

(57) ABSTRACT

The invention concerns the use of VCAM-1 ligands in medical imagery, in particular for characterizing and/or therapeutic monitoring of cardiovascular diseases and more particularly for detecting vulnerable coronary atheroma. The invention also concerns the use of VCAM-1 ligands for making a medicine for treating a cardiovascular disease.

The invention relates to the use of VCAM-1 ligands in medical imaging, in particular for characterizing and/or therapeutic monitoring of cardiovascular diseases and more particularly for detecting vulnerable coronary atheromatous plaque. The invention also relates to the use of VCAM-1 ligands for manufacturing a medicament intended for treating a cardiovascular disease.

Cardiovascular diseases, and in particular myocardial infarction, represent the prime cause of mortality in industrialised countries. Atherosclerosis is the principal causal factor in coronary diseases.

The process leading to the development of an atheromatous plaque involves an initial lipidic infiltration of the intima of an artery, marked by the passive penetration and the accumulation of low-density lipoproteins (LDL-cholesterol) followed by an oxidative modification of these LDL. This mechanism favours the adhesion of the monocytes circulating in the region of the surface of the endothelium. These monocytes penetrate the sub-endothelial space and are transformed into macrophages which then bring about a local chronic inflammatory reaction and the production of pro-inflammatory cytokines.

The atheromatous plaque comprises a lipidic core and a fibrous shell, which consists of smooth muscular cells, collagens and an extracellular matrix and isolates the lipidic core of the arterial lumen. When the plaque develops and protrudes into the arterial lumen it brings about the formation of an arterial stenosis which can induce an ischaemia.

The principal risk is the rupture of the plaque which occurs in the region of the fibrous shell and puts the blood into contact with the thrombogenic elements of the lipidic core, thus exposing the subject to an arterial thrombosis. In particular, the rupture of a vulnerable coronary atheromatous plaque is the cause of the majority of coronary events (myocardial infarction, sudden death, unstable angina). The vulnerability of the plaque, that is to say its propensity to rupture, results essentially from the size of the lipidic core and/or a weakening of the fibrous shell, for example by inflammatory mechanisms.

The vulnerable coronary atheromatous plaque, which is characterized by a substantial soft lipidic core, a thin fibrous shell and exacerbated inflammation, is invisible to coronarography but is studied perfectly in an invasive manner by intra-coronary ultrasound imaging. However, there is currently no atraumatic method of imaging which makes it possible to reveal these vulnerable coronary atheromatous plaques.

Magnetic resonance imaging is a non-invasive technique which has been used for the imaging of carotid plaques. However, its resolution does not enable imaging of coronary atheromatous plaques. Another non-invasive technique is nuclear medicine: it enables for example the realisation of scintigraphic images after injection of specific radioactive tracers of a biological target. However, currently no radioactive tracer exists which can be used in nuclear medicine for the detection of vulnerable plaques. Numerous targets have been studied (LDL-Tcm^{99m}, LDL-Iodine¹²³, antireceptor antibody Fc of macrophages, etc. . . . ) but none is sufficiently specific for vulnerable atheromatous plaques (Narula et al., 1999; Fayad and Fuster, 2001).

The development of specific radioactive tracers for vulnerable coronary atheromatous plaque should enable simple discovery, by external detection of the radioactivity, of the presence and the number of vulnerable plaques. Such detection is a major opportunity for predicting the occurrence of coronary events in a patient and for being able to adapt the therapy as a function of the risk of coronary events in this patient.

The existence of an inflammatory phenomenon is a characteristic of the vulnerable plaque. In particular it has been shown that VCAM-1, an endothelial adhesion molecule involved in the adhesion of monocytes to the vascular endothelium, is overexpressed on the surface of the vulnerable coronary atheromatous plaques. However, VCAM-1 has never been chosen as target for the development of a radioactive tracer for the vulnerable atheromatous plaque.

The inventors have shown that radioactive VCAM-1 ligands constitute specific tracers for the vulnerable coronary atheromatous plaque and enable imaging thereof by simple external detection of the radioactivity after intravenous injection.

Tracers have in particular been developed on the basis of the peptide B2702 consisting of the residues 75 to 84 of the molecule HLA-B2702. This peptide (B2702.84-75) as well as the peptide consisting of the repeated motif 75-84 (B2702.84-75/75-84) bind specifically to VCAM-1 as shown by Ling et al. (2000).

After synthesis and radioactive labelling, the tracers were evaluated on animal models used for the study of the development of atherosclerosis. The results of the inventors show that, after injection in vivo, the labelled VCAM-1 ligands are bound specifically on the atheromatous plaques which develop on the aortas of hyperlipidemic rabbits.

Therefore these results indicate that contrast agents containing a VCAM-1 ligand are useful in medical imaging, in particular for the detection of a cardiovascular disease in a patient. The ability to detect and monitor the formation of an atherosclerotic plaque, more specifically a vulnerable coronary atheromatous plaque, is a major challenge bearing in mind the role which these plaques play in the occurrence of acute ischaemic syndromes. The use of contrast agents according to the invention is therefore particularly useful for the detection of vulnerable plaques in a population exhibiting a cardiovascular risk. Furthermore these contrast agents can be used in order to establish a prognostic index which makes it possible to adapt a therapeutic treatment to the risks of the patient.

### DEFINITIONS

“VCAM-1” (Vascular Cell Adhesion Molecule 1) designates a cell adhesion protein of which the transcription is induced in endothelial cells but which is also expressed by other cell types. VCAM-1 was discovered and cloned by Osborn et al. in 1989. VCAM-1 interacts with integrin α4β1, also known as VLA-4 (Very Late Antigen 4), which is expressed as a component by lymphocytes and monocytes, in particular. Like other adhesion molecules, such as ICAM-1, 2, and 3, VCAM-1 is involved in the adhesion of monocytes to the endothelium in the course of the atherosclerosis. VCAM-1 also interacts with integrin α4β7 for the recruitment of lymphocytes in the region of the intestine.

“VCAM-1 ligand” is understood to mean a molecule capable of binding selectively to VCAM-1. The ligand is preferably specific to VCAM-1, that is to say that it binds to VCAM-1 to the exclusion of any other molecule. A ligand can be a small molecule such as a chemical compound, or a large molecule such as polypeptides or proteins, including antibodies, growth factors or integrins. Examples of VCAM-1 ligands of a polypeptide nature include the integrins α4β1 and α4β7, or also the peptides B2702.84-75 and B2702.84-75/75-84, or the compound 4(B 2702.84-75)Raft. The VCAM-1 ligands also include aptamers.

“Aptamers” constitute a class of molecules which represent an alternative to the antibodies in terms of molecular recognition. Aptamers are oligonucleotide sequences having the capacity to recognise virtually any class of target molecules whatsoever with a high affinity and specificity. Such ligands can be isolated by screening known as SELEX (Systematic Evolution of Ligands by EXponential enrichment) of a library of random sequences, as described in Tuerk and Gold (1990). The random sequence library can be obtained by synthesis of DNA by combinatorial chemistry. In such a library each member is a linear oligomer, optionally chemically modified, corresponding to a unique sequence. The possible modifications, applications and advantages of this class of molecules are the subject of a review by Jayasena (1999). Aptamers may also be of a peptide nature. They comprise a variable region which is structurally constrained, assembled on a protein framework, such as Thioredoxin A of E. coli, and can be selected from combinatorial libraries by double hybrid methods (Colas et al., 1996).

The peptide “B2702.84-75” designates the peptide of sequence YRLAIRLNER (SEQ ID n^{o}1), which is constituted by the inverse sequence of the 10 C-terminal residues (residues 84 to 75) of the alpha helix α1 of the molecule HLA-B2702 (Ling et al., 2000).

The peptide “B2702.84-75/75-84” designates the peptide of sequence YRLAIRLNERRENLRIALRY (SEQ ID n^{o}2), constituted by an inverse dimer of the 10 C-terminal residues of the alpha helix al of the molecule HLA-B2702 (residues 84-75/75-84) (Ling et al., 2000).

The compound 4(B 2702.84-75)Raft designates a molecule constituted by a cyclopeptide framework ₁GKAKPGKKKP₁₀ (“Raft”) bearing on one and the same face four peptides B 2702.84-75 grafted on the lysines in position 2, 4, 7 and 9 of the decapeptide. The cyclopeptide framework bears on its other face a group Z, grafted via the lysine in position 8, and designates any contrast agent which enables imaging of the binding between VCAM-1 and the compound 4(B 2702.84-75)Raft. The structure of the compound 4(B 2702.84-75)Raft is shown in FIG. 2. The synthesis and the grafting of the cyclopeptide framework can be effected in accordance with the process described in the international application WO 2004026894.

[^{99m}Tc]-B 2702.84-75/75-84, [^{99m}Tc]-B 2702.84-75 or [^{99m}Tc]-4(B 2702.84-75)Raft designate a peptide B 2702.84-75/75-84, B 2702.84-75 or 4(B 2702.84-75)Raft, which is labelled by technetium 99 and of which the peptide sequence has optionally been modified, for the purposes of labelling, by the addition of one, two or more amino acid(s), in particular by the addition at least of a histidine at the C-terminal or N-terminal end of the peptide sequence. For example, the peptide [^{99m}Tc]-B 2702.84-75 preferably designates the peptide of sequence YRLAIRLNERGH (SEQ ID n^{o}3) in which technetium 99 is bound to histidine.

Within the context of the present invention, a “cardiovascular disease” designates a disease, a lesion or a symptom linked to a process of atherogenesis affecting the cardiovascular system. This includes in particular the states which mark the development of an atheromatous plaque (the plaques are classified into stages of progression I to VI, according to the international Stary classification), as well as the complications ensuing from the formation of an atheromatous plaque (stenosis, ischaemia) and/or from its evolution towards an acute ischaemic event (thrombosis, embolism, infarction, arterial rupture). Cardiovascular diseases designate for example atherosclerosis, an atheromatous plaque, in particular a vulnerable plaque, coronary disease, angina, thrombosis, cerebral vascular accident, myocardial infarction, vascular stenosis, infarction.

The “Coronary disease” is the most common manifestation of cardiovascular disease. It is a progressive disease, due to poor supply of the cardiac muscle, subsequent to the narrowing (stenosis) or the calcification (sclerosis) of a coronary artery or the coronary arteries. The principal symptom of coronary disease manifests itself in the form of pains which constitute angina (stable or instable), also known as angina pectoris. The complete obstruction of a coronary artery of the coronary arteries leads to the infarction.

“Infarction” designates a circumscribed seat of necrosis due to an arterial obstruction. More specifically, myocardial infarction is a necrosis of the myocardium which results generally from an acute coronary thrombosis, secondary to a rupture of plaque (generally an unstable plaque or vulnerable plaque) giving rise to the aggregation of platelets then to coronary occlusion.

“Thrombosis” corresponds to the coagulation of blood in the vascular cavities (arteries, veins, capillaries or cardiac cavities) leading to the formation of a thrombus.

“Embolism” is the intravascular migration of a foreign body, most often formed by a blood clot (thrombus), which stops suddenly in a vessel of which the size is insufficient to allow it to pass through. The local consequences of the embolism are circulatory disturbances linked to the vascular obstruction, most often leading to an infarction.

“Ischaemia” designates the decrease in the arterial blood supply in an area of the organism. The principal local causes thereof are thrombosis and embolism.

The expression “vulnerable plaque” designates an atheromatous plaque having a thin fibrous shell (approximately 65 to 150 μm thick) and a substantial lipidic core. These unstable plaques, which have a tendency to rupture, are observed in the region of the coronary arteries and in the region of the aorta and of its branches. The rupture of the vulnerable coronary plaques causes “acute coronary syndromes”: in the event of complete occlusive thrombosis, this involves myocardial infarction; when the thrombosis of the artery remains incomplete it involves unstable angina. In the region of the carotid, the vulnerable plaques are more stenotic and less inflammatory. They also express VCAM-1.

A “contrast agent” designates a substance or a composition which, administered in the organism, enables detectable labelling of the organs or of the structures (tissue, cell, receptor) which, without contrast agent, are only slightly visible or are invisible in medical imaging. By extension, the expression “contrast agent” is used for designating a tracer associated with a marker.

“Tracer” is generally understood to mean a substance which can be located selectively in the region of a particular structure of the organism (tissue, organ, cell, receptor, for example). It may be a simple element (an atom), a molecule (for example a protein), or a more complex structure (liposome, cell, etc. . . . ). Within the context of the present application the tracer is a VCAM-1 ligand.

“Marker” is understood to mean a compound which produces a detectable signal. When it is associated with a tracer, it makes it possible to follow the evolution of the tracer in the organism. The marker may be a MRI contrast agent, a scintigraphy contrast agent, an X-ray imaging contrast agent, an ultrasound contrast agent or an optical imaging contrast agent.

### Contrast Agent

The inventors have demonstrated that VCAM-1 ligands constitute specific tracers for atheromatous plaque, in particular for aortic and/or vulnerable coronary plaque, and enable detection thereof by imaging.

Therefore the invention proposes the use of a VCAM-1 ligand for the production of a contrast agent which is useful for medical imaging. However, the medical imaging technique which can be used is preferably a non-invasive in vivo imaging technique.

The VCAM-1 ligand is preferably selected from within the group comprising the peptide B 2702.84-75/75-84, the peptide B 2702.84-75, the compound 4(B 2702.84-75)Raft, and VCAM ligand aptamers.

The VCAM-1 ligand can also be a derivative of the peptides B 2702.84-75 or B 2702.84-75/75-84, or a derivative of the compound 4(B 2702.84-75)Raft. A “derivative” of these peptides or of this compound is understood to be a peptide or compound in which the sequence RENLRIALRY constituting the sequence 84-75 of the peptide B 2702 has been modified by addition, deletion, substitution or modification of at least one amino acid. The sequence RENLRIALRY can in particular be modified by substitution of one, two or three amino acids. The substitution may or may not be conservative. The substitution is conservative when an amino acid is substituted by an amino acid having similar properties (for example polarity, hydrogen bonding potential, acidity, basicity, hydrophobicity, presence of an aromatic group, etc. . . . ). Amino acids having similar properties are well known to the person skilled in the art, for example arginine, histidine and lysine are hydrophilic-basic amino acids which can be interchangeable; isoleucine, a hydrophobic amino acid, can be replaced by leucine, methionine or valine. A natural amino acid can be replaced by a non-natural amino acid, such as a D configuration amino acid, beta or gamma amino acid. The sequence RENLRIALRY can be modified by replacing one or several amide bonds by a bond giving an increased stability in vivo, for example giving an increased resistance to proteolysis.

A derivative of the compound 4(B 2702.84-75)Raft may have one, two, three or four derivative peptides B 2702.84-75, and the said derivative peptides B 2702.84-75 grafted on the cyclic framework may or may not be modified in an identical manner.

The derivative peptides B 2702.84-75 or B 2702.84-75/75-84, or the derivative compound 4(B 2702.84-75)Raft retain the ability to bind VCAM-1. These derivatives preferably have an affinity for binding with VCAM-1 which is equal or increased with respect to the corresponding peptide or compound, B 2702.84-75, B 2702.84-75/75-84 or 4(B 2702.84-75)Raft. The measurement of the affinity of binding of a ligand with VCAM-1 can be carried out for example by using the technique of fluorescence polarization (Checovich et al., 1995).

The principle of fluorescence polarization is based on the study of the polarization of the light emitted by a compound marked with a fluorophore (such as fluorescein for example). When it is in solution, a fluorescent compound is in rotation at a constant speed which is dependent upon the mass of the ligand and the viscosity of the medium. When subjected to luminous excitation, it is possible for this compound to reflect a depolarized light, and to do so in all spatial directions. The reflected light is analyzed with the aid of two fixed sensors separated by an angle of 90°. These sensors make it possible to collect the re-emitted light in a reception plane and to express two values (the anisotropy and the polarisation) on the basis of the collected intensities from the beams parallel or perpendicular to the excitation beam. The anisotropy and the polarization are two factors linked to one another. If a ligand is added to the solution containing a fluorescent compound, the luminous properties of the fluorescent compound change in the compound/ligand complex and the re-emitted light becomes polarized. The anisotropy represented as a function of the concentration of ligand in solution evolves according to a sigmoid curve, with a phase of progressive increase in the relative intensity of fluorescence linked to the increasing addition of ligand and thus to the appearance of the complex (compound/ligand), followed by a high plateau reflecting the presence of the complex which polarizes the light at a speed of rotation in solution which is slowed down with respect to the fluorescent compound alone. The measurements of fluorescence polarization can be carried out for example with a PERKIN ELMER LS50 luminescence spectrometer. For the measurement of the affinity of VCAM-1 ligands the compound marked by the fluorophore may be either the VCAM-1 ligand or VCAM-1. It is then possible to compare the affinities of different VCAM-1 ligands for VCAM-1 by determining graphically the concentration of ligand (if VCAM-1 is the marked fluorescent compound) necessary for forming 50% of the VCAM-1/VCAM-1 ligand complexes.

The use of a VCAM-1 ligand for the production of a contrast agent is more particularly for diagnosis of a cardiovascular disease by medical imaging.

The medical imaging techniques used for diagnosing a cardiovascular disease, and in particular for detecting atherosclerotic plaques, bring together invasive techniques such as angiography or coronarography, endocoronary ultrasound scanning, or non-invasive techniques such as doppler velocimetry, angiography by magnetic resonance imaging or nuclear medicine. The nuclear medicine techniques more particularly used in the cardiovascular field include perfusion scintigraphy.

The use of a contrast agent, chosen as a function of the imaging technique used (MRI contrast agent, scintigraphy contrast agent, X-ray imaging contrast agent, ultrasound contrast agent, optical imaging contrast agent), make it possible to target a tissue, an organ or a pathological area in a specific manner.

Scintigraphy, for example, is based on the administration (generally by the intravascular route) of a contrast agent, also known as a radio-pharmaceutical agent, consisting of a tracer labelled by radioactive isotope. The specific location of this contrast agent in the organism is then determined by detection of the gamma or beta rays emitted.

VCAM-1 ligands can be used with different imaging techniques. By way of examples, imaging techniques and associated markers which can be used include TEP imaging, the marker preferably being fluorine 18 (¹⁸F); MRI, the markers preferably being nanoparticles of iron oxide or of gadolinium; fluorescence imaging, the marker preferably being fluorescein, Alexa or cyanine; chemiluminescence imaging, the marker preferably being luminol; bioluminescence imaging, the marker being luciferase or alkaline phosphatase in particular. Optical imaging brings together the fluorescence, chemiluminescence and bioluminescence imaging techniques.

The marker is preferably a radioactive nuclide. Examples of radioactive nuclides most used in nuclear imaging include technetium 99m (^{99m}Tc), iodine 123 (¹²³I), indium 111 (^{111m}In), fluorine 18 (¹⁸F), gallium 67 (⁶⁷Ga), iodine 125 (¹²⁵I), or any other radioactive nuclide which can be used in humans. The marker can be bound to the tracer by substitution (for example when the ligand is a protein, by substituting an H by an I in the region of the tyrosine residues), by complexing or by chelation.

Therefore the VCAM-1 ligand is advantageously associated with a marker which makes it possible to localise or to facilitate location of the VCAM-1 ligand in an organism to which the said contrast agent has been administered. The marker can be selected from within the group comprising a radioactive nuclide such as ¹²³I, ¹²⁵I, ^{99m}Tc, ¹¹¹In, ¹⁸F, ⁶⁷Ga, a fluorophore such as fluorescein, Alexa, cyanine, a chemiluminescent compound such as luminol, a bioluminescent compound such as luciferase, alkaline phosphatase, or an MRI contrast agent such as nanoparticles or gadolinium. The choice of the appropriate marker, which is ultimately a function of the medical imaging technique used, is within the capacity of the person skilled in the art. According to a preferred embodiment, the VCAM-1 ligand is labelled by a radionuclide.

The said labelled VCAM-1 ligand is also preferably selected from within the group comprising [¹²⁵I]-B 2702.84-75/75-84, [¹²⁵I]-B 2702.84-75, [¹²³I]-B 2702.84-75/75-84, [¹²³I]-B 2702.84-75, [^{99m}Tc]-B 2702.84-75/75-84, [^{99m}Tc]-B 2702.84-75, [¹²³I]-4(B 2702.84-75)Raft, [¹²⁵I]-4(B 2702.84-75)Raft, and [^{99m}Tc]-4(B 2702.84-75)Raft, VCAM ligand aptamers labelled with iodine 123, iodine 125 or Tc99m. Medical imaging in the sense of the invention may be a nuclear imaging, MRI or optical imaging technique. Medical imaging is preferably a nuclear imaging technique such as scintigraphy.

The invention also relates to a contrast agent comprising a VCAM-1 ligand, optionally labelled, as described above.

### Use of VCAM-1 Ligands

The appearance of atheromatous plaques is a sign of atherosclerosis, which itself constitutes a cardiovascular disease and can generate various cardiovascular complications. The detection of atheromatous plaques therefore constitutes the characterization of a cardiovascular disease. Moreover, the possibility of using imaging to monitor the evolution, that is to say the progression or the regression, of a previously identified atheromatous plaque represents a way of evaluating the efficacy of a therapeutic treatment in a patient in whom a cardiovascular disease has been diagnosed.

According to one embodiment, the invention thus relates to the use of a VCAM-1 ligand for the production of a contrast agent which can be used for the detection of a cardiovascular disease and/or a risk of an acute ischaemic event in a subject likely to exhibit such a disease.

The contrast agent is preferably used for detecting atherosclerosis, in particular for detecting an atheromatous plaque. It may be a vulnerable atheromatous plaque.

More specifically the said plaque may be a vulnerable coronary atheromatous plaque. The use of a VCAM-1 ligand for the production of a contrast agent is therefore useful for the detection of coronary disease, in particular stable or unstable angina. Moreover, the presence of a coronary atheromatous plaque, especially if it is a vulnerable plaque, exposes the subject to a risk of an acute ischaemic event, in particular a myocardial infarction. The use of a VCAM-1 ligand for the production of a contrast agent is therefore useful for the detection of a risk of myocardial infarction in a subject.

The atheromatous plaque can also be located in the region of a carotid artery (carotid atheromatous plaque). These lesions lead to cerebral vascular accidents, haemorrhagic events (rupture of aneurism) or ischaemic events (cerebral infarction). The use of a VCAM-1 ligand for the production of a contrast agent is therefore equally useful for the detection of a risk of cerebral vascular accident in a subject.

The atheromatous plaque may also be located in the region of a renal artery, the kidney being one of the organs targeted by atherosclerosis. A substantial stenosis can lead to arterial hypertension and/or to renal insufficiency. The atheromatous attack on the renal arteries can lead to an acute vascular accident, a renal embolism. Therefore the invention also relates to the use of a VCAM-1 ligand for the production of a contrast agent which can be used for the detection of arterial hypertension and/or renal insufficiency, and/or for the detection of a risk of renal embolism in a subject.

Atheromatous plaques can be located in the region of the arteries of the lower limbs (risk of acute ischaemia of a limb), or of the aorta (risk of rupture of aneurism/aortic dissection). The invention then relates to the use of a VCAM-1 ligand for the production of a contrast agent which can be used for the detection of a risk of acute ischaemia of a limb or of rupture of an aortic aneurism in a subject.

According to one embodiment, the invention relates to the use of a VCAM-1 ligand for the production of a contrast agent which can be used for therapeutic monitoring of a cardiovascular disease in a subject in whom a cardiovascular disease has been diagnosed.

Therapeutic monitoring is understood to mean the observation of the response of the subject to the treatment administered to it. The therapeutic effect of a treatment is generally associated with a slowing down or an inhibition of the progression of a disease, a reversal of the disease, or one or several symptoms associated with this disease. Conversely, an absence of therapeutic effect may be manifested by a stability, or indeed an acceleration, of the progression of the disease or of one or several of its symptoms.

For example, if the cardiovascular disease in the sense of the invention is an atheromatous plaque, the therapeutic monitoring can be carried out by observing the disappearance, the regression, the maintenance or the growth of the atheromatous plaque. Thus the use according to the invention can comprise the steps of:

a) administering a VCAM-1 ligand to a subject in which an atheromatous plaque has been detected;

b) detecting the binding of the VCAM-1 ligand in the region of the said atheromatous plaque;

c) repeating steps a) and b) before and after administration of a treatment to the said subject;

an absence or a diminution of the binding of the VCAM-1 ligand in the region of the said plaque being indicative of a treatment having a therapeutic effect.

Within the context of the present application, a “subject” denotes a human or non-human mammal, such as a rodent (rat, mouse, rabbit), a primate (chimpanzee), a feline (cat) or a canine (dog). The subject according to the invention is preferably a human.

### Method of Diagnosis

The invention also relates to a method of diagnosis of a cardiovascular disease and/or of detection of a risk of an acute ischaemic event in a subject likely to exhibit a cardiovascular disease, the said method comprising the steps of administering a VCAM-1 ligand to the said subject and detecting the said VCAM-1 ligand in the organism of the said subject, the detection of a preferred location of the said VCAM-1 ligand in the region of the cardiovascular system being indicative of a cardiovascular disease and/or of a risk of an acute ischaemic event.

“Preferred location” is understood to mean that the quantity of VCAM-1 ligand detected in the region of the cardiovascular system is greater than the background noise which corresponds to a non-specific location of the VCAM-1 ligand in the organism.

Preferably, the VCAM-1 ligand is selected from within the group consisting of the peptide B 2702.84-75/75-84, the peptide B 2702.84-75, the compound 4(B 2702.84-75)Raft, and VCAM ligand aptamers.

The VCAM-1 ligand is advantageously associated with a marker which makes it possible to locate or to facilitate the location of the VCAM-1 ligand in an organism to which the said contrast agent has been administered.

The marker can be selected from within the group consisting of a radioactive nuclide such as technetium 99m (^{99m}Tc), iodine 123 (¹²³I), indium 111 (^{111m}In), fluorine 18 (¹⁸F), gallium 67 (⁶⁷Ga), iodine 125 (¹²⁵I), or any other radioactive nuclide which can be used in humans, a fluorophore such as fluorescein, Alexa or cyanine, a chemiluminescent compound such as fluorescein, Alexa or cyanine, a chemiluminescent compound such as luminol, a bioluminescent compound such as luciferase or alkaline phosphatase and an MRI contrast agent such as nanoparticles or gadolinium. According to a preferred embodiment, the VCAM-1 ligand is labelled by a radioactive nuclide.

The labelled VCAM-1 ligand is preferably selected from within the group consisting of [¹²⁵I]-B 2702.84-75/75-84, [¹²⁵I]-B 2702.84-75, [¹²³I]-B 2702.84-75/75-84, [¹²³I]-B 2702.84-75, [^{99m}Tc]-B 2702.84-75/75-84, [^{99m}Tc]-B 2702.84-75, [¹²³I]-4(B 2702.84-75)Raft, [¹²¹I]-4(B 2702.84-75)Raft, and [^{99m}Tc]-4(B 2702.84-75)Raft, VCAM ligand aptamers labelled with iodine 123, iodine 125 or Tc99m.

The VCAM-1 ligand can be detected by any means known to the person skilled in the art. In particular, the choice of an appropriate imaging technique as a function of the type of marker optionally associated with the VCAM-1 ligand is within the capacity of the person skilled in the art. The location of the VCAM-1 ligand is advantageously effected by means of a non-invasive technique such as nuclear imaging, in particular scintigraphy, or MRI.

The VCAM-1 ligand can be administered for example orally, by inhalation, parenterally (in particular by intravenous injection), in an appropriate form. When the parenteral route is envisaged, the VCAM-1 ligand can be in the form of solutes and injectable suspensions packaged in ampoules or vials. The forms for parenteral administration are obtained conventionally by mixing the VCAM-1 ligand with buffers, stabilising agents, preservatives, solubilising agents, isotonic agents and suspension agents. According to known techniques, these mixtures are then sterilised and subsequently packaged in the form of intravenous injections. By way of a buffer, the person skilled in the art will be able to use buffers based on organic phosphate salts. Examples of suspension agents encompass methylcellulose, hydroxyethylcellulose, hydroxy-propylcellulose, acacia and sodium carboxymethylcellulose. Moreover, stabilisers which can be used according to the invention are sodium sulphite and sodium metasulphite, whilst mention may be made of sodium p-hydroxybenzoate, sorbic acid, cresol and chlorocresol as preservatives.

The quantity of VCAM-1 ligand administered naturally depends upon the mode of administration, the size and/or the weight of the patient, and the detection technique used.

For example the dose of ligand used for nuclear imaging is preferably between approximately 20 and approximately 500 micrograms.

### Method of Treatment

The invention also relates to the use of a VCAM-1 ligand for the production of a medicament intended for the treatment of a cardiovascular disease. A method of treatment of a cardiovascular disease comprising the administration of a therapeutically effective quantity of a VCAM-1 ligand also forms part of the present invention.

The use of a ligand of VCAM-1, coupled with a cytotoxic agent, makes it possible in effect to selectively destroy the inflammatory cells which express VCAM-1 and which contribute to the instability of the atherosclerotic plaque.

The “treatment” of a cardiovascular disease is understood to mean the “therapeutic treatment” (or curative treatment) of a cardiovascular disease, which includes the slowing down or the inhibition of the evolution of an atherosclerotic plaque, in particular towards a vulnerable atherosclerotic plaque stage, or the regression of an atherosclerotic plaque, in particular of a vulnerable plaque; it is also understood to mean the “prophylactic treatment” of a cardiovascular disease which includes in particular the prevention of an acute ischaemic event.

The VCAM-1 ligand is preferably selected from within the group consisting of the peptide B 2702.84-75/75-84, the peptide B 2702.84-75, the compound 4(B 2702.84-75)Raft, and VCAM ligand aptamers.

The VCAM-1 ligand is advantageously coupled with a cytotoxic agent in such a way as to put the cytotoxic agent in contact with a cell expressing VCAM-1 and thus to selectively destroy the latter. The choice of an appropriate cytotoxic agent is within the capacity of the person skilled in the art. Examples of cytotoxic agents include in particular radioactive isotopes, toxins or chemotherapeutic agents such as doxorubicin or taxotere, for example.

According to one embodiment the VCAM-1 ligand is coupled with a radioactive isotope in such a way as to carry out a curative and/or preventive radiotherapy of a cardiovascular disease. The radiotherapy must use radiation of which the average path is weak in order to deposit the majority of its energy upon contact with the tissue or the target cells. This is why the radioactive isotope is preferably chosen from among the β⁻ emitters, such as iodine 131 (¹³¹I) and yttrium 90 (⁹⁰Yt).

The method of treatment according to the invention can advantageously be carried out using a VCAM-1 ligand coupled with a radioactive isotope selected from within the group consisting of [¹³¹I]-B 2702.84-75/75-84, [¹³¹I]-B 2702.84-75, [⁹⁰Yt]-B 2702.84-75/75-84, [⁹⁰Yt]-B 2702.84-75, [¹³¹I]-4(B 2702.84-75)Raft, [⁹⁰Yt]-4(B 2702.84-75)Raft, and VCAM ligand aptamers labelled with iodine 131 or yttrium 90.

According to another embodiment, the VCAM-1 ligand can be coupled with a toxin, for example ricin or a bacterial toxin, such as saporin or the cholera toxin.

The invention relates more particularly to the use of a VCAM-1 ligand for the production of a medicament intended for the treatment of atherosclerosis and/or for the prevention of an acute ischaemic event in a subject likely to exhibit a cardiovascular disease.

The VCAM-1 ligand is preferably used for treating a vulnerable atheromatous plaque, even more preferably for a vulnerable coronary or aortic atheromatous plaque.

The said acute ischaemic event is preferably selected from within the group consisting of a myocardial infarction, a cerebral vascular accident, a renal embolism, an acute ischaemia of a limb, and a rupture of an aortic aneurism.

The VCAM-1 ligand can be administered for example orally, by inhalation, parenterally (in particular by intravenous injection), in an appropriate form. When the parenteral route is envisaged, the VCAM-1 ligand can be in the form of solutes and injectable suspensions packaged in ampoules or vials. The forms for parenteral administration are obtained conventionally by mixing the VCAM-1 ligand with buffers, stabilising agents, preservatives, solubilising agents, isotonic agents and suspension agents. According to known techniques, these mixtures are then sterilised and subsequently packaged in the form of intravenous injections. By way of a buffer, the person skilled in the art will be able to use buffers based on organic phosphate salts. Examples of suspension agents encompass methylcellulose, hydroxyethylcellulose, hydroxy-propylcellulose, acacia and sodium carboxymethylcellulose. Moreover, stabilisers which can be used according to the invention are sodium sulphite and sodium metasulphite, whilst mention may be made of sodium p-hydroxybenzoate, sorbic acid, cresol and chlorocresol as preservatives.

The quantity of VCAM-1 ligand administered naturally depends upon the mode of administration, the size and/or the weight of the patient, and the detection technique used. For example when the VCAM-1 ligand is labelled, for example with iodine 131, it is possible to administer approximately 25 mg of VCAM-1 ligand per m² of body surface, which corresponds to an injected activity of 50 mCi of iodine 131.

The following examples and drawings illustrate the invention without limiting the scope thereof.

### DESCRIPTION OF THE DRAWINGS

FIG. 1 shows the results of analysis of the tissue biodistribution of the tracers [¹²⁵I]-B2702.84-75/75-84 and [¹²⁵I]-B2702.84-75 in the WHHL hyperlipidemic rabbit.

FIG. 2 shows the structure of the compound 4(B 2702.84-75)Raft.

FIG. 3 shows the aortic activity of ¹²³I-B2702.84-75 (¹²³I-B2702-p) and ^{99m}Tc-B2702.84-75 (^{99m}Tc-B2702-p) standardised to the blood activity, 180 minutes after the injection of the tracers in control and WHHL animals. * P<0.05 vs. Controls.

FIG. 4 shows the blood kinetics of ¹²³I-B2702.84-75 and ^{99m}Tc-B2702.84-75.

FIG. 5 shows the quantification of the aortic activities of ¹²³I-B2702.84-75 (A) and ^{99m}Tc-B270284-75 (B) on the basis of the ex vivo autoradiographic images produced 180 minutes after the injection of the tracers in the control animals and in the healthy zones (Sudan IV-negative) or the zones of lipidic accumulation (Sudan IV-positive) of the WHHL animals. * P<0.05 vs. Controls, t P<0.5 vs. Sudan IV-negative zones.

### EXAMPLE 1

### Material and Methods

The specificity of the VCAM-1 ligands with respect to vulnerable atheromatous plaques was studied on an animal model.

Animal Model

The biological model used for the evaluation of the capacity of VCAM-1 ligands to bind to the atheromatous plaques is the genetically hyperlipidemic rabbit of the WHHL (Watanabe Heritable Hyperlipidemic) strain, which represents a model of choice for the development of atherosclerosis (Clubb et al. (2001)). The animals were obtained from Centre de Production Animale (Olivet, France).

Synthesis of Peptides

The VCAM-1 ligand chosen for this study is the molecule HLA-B2702. Ling et al. (2000) have shown that the motif consisting of the residues 75 to 84 of HLA-B2702 (B2702.84-75) as well as this same repeated motif (B2703.84-75/75-84) bind specifically to VCAM-1.

The peptides B2702.84-75 [YRLAIRLNER], B2703.84-75/75-84 [YRLAIRLNERRENLRIALRY] and a peptide B2702.84-75 modified for labelling with technetium, B2702-His [YRLAIRLNERGH] were prepared by synthesis in the solid phase. The assemblage protected peptides were assembled either manually, using a Fmoc/tBu strategy in a glass reactor equipped with a glass frit, or automatically on a synthesizer (348Ω synthesizer, Advance ChemTech). The coupling reactions were effected using 1.5-2 eq. amino acid protected by N-α-Fmoc, activated in situ with 1.5-2 eq. of PyBOP and 3-4 eq. of DIEA in DMF (10 ml/g of resin) for a period of 30 min (the proportions are given relative to the amount of resin). With manual coupling, the syntheses are monitored by Kaiser tests and/or TNBS tests. The protective N-α-Fmoc groups were removed by treatment with a solution of piperidine:DMF (1:4) (10 ml/g of resin) for 10 min. The treatment was repeated three times and the complete deprotection was monitored by measurement of the UV absorption at 299 mm of the piperidine washes. The linear synthetic peptides were collected directly after acid cleavage (1% TFA in CH₂Cl₂). The resins were treated for 3 min repeatedly until the beads of resins turn dark violet. The washes were collected and concentrated under reduced pressure and solid white peptides were obtained by precipitation with ether then analysed by RP-HPLC.

Radiolabelling

B2703.84-75/75-84 was labelled with ¹²⁵I, (Amersham Radiochemical Center) and B2702.84-75 with ¹²⁵I or ¹²³I (Schering SA) on tyrosine (amino acid in position 84) using chloramine-T. Briefly, 74 MBq of ¹²⁵I or 200 MBq of ¹²³Iwere added to 20 or 40 nmoles of peptide and 20 or 80 μl of chloramine T prepared extemporaneously (1 mg/ml), respectively. The reaction was stopped after 15 minutes by addition of 20 μl or 80 μl of sodium metabisulphite (4 mg/ml) for ¹²⁵I and ¹²³I, respectively. Analysis by thin layer chromatography (TLC) (RP-18R254; Merck) with an acetonitrile/H₂O mixture (60/40) as eluent enables evaluation of the radiochemical purity.

Two amino acids [HG] were added to the peptide B2702.84-75 for labelling by ⁹⁹mTc on histidine with [^{99m}Tc(OH₂)₍₃₎ (CO)₍₃₎] using a tridentate ligand system. The precursor [^{99m}Tc(OH₂)₍₃₎(CO)₍₃₎] was synthesized using a “tricarbonyl pharmaceuticals Kit” (Isolink®, Mallinckrodt). The kit was reconstituted with 2 GBq of ^{99m}TcO₄⁻ (Schering SA), and incubated at 100° C. for 20 minutes. After adjustment of the pH to 8.0, 800 MBq of this solution were added to 30 nmoles of modified B2702.84-75 and incubated for 20 minutes at 80° C.

Experimental Protocol

For the experiments described above B2702.84-75/75-84 and B2702.84-75, labelled with iodine-125 ([¹²⁵I]-B2702.84-75/75-84 and [¹²⁵I]-B2702.84-75) by electrophilic substitution, are injected intravenously (˜120 μCi/kg) into normal or hyperlipidemic animals (n=4/group) of comparable weight and age previously anaesthetized by intramuscular injection of a xylazine (5 mg/kg)/ketamine (35 mg/kg) mixture and catheterised in the controlateral veins of the ear.

Samples of blood were taken 1, 2, 5, 10, 15, 20, 25 and 30 minutes after injection of the tracers. The animals were euthanased 30 minutes (examples 2, 3 and 4) or 180 minutes (examples 5 and 6) after injection of the labelled VCAM-1 ligands. Samples of the principal organs were taken. These samples, as well as the blood samples taken, were counted for the radioactivity which they contain. The dorsal aorta, the aortic arch and the ventral aorta were also sampled and immediately placed in 10% formalin. A portion of ventral aorta was placed for 24 hours in 3.7% formaldehyde for subsequent inclusion in paraffin and verification by immunohistology of the presence of VCAM-1.

The dorsal and ventral aortas were placed in contact with a high-resolution phosphorus screen (Fujifilm 08SR2025) in darkness for 18 hours. The screen was then scanned (phosphoimager Fujifilm BAS 5000) and the images of the distribution of the labelled VCAM-1 ligands on the aortas of healthy or hyperlipidemic animals were quantified with the aid of the Scion Image Beta software available on the National Institute of Health internet site.

Finally, staining with Sudan IV was carried out on the dorsal and ventral aortas and enabled comparison of the preferred regions for binding of the tracers with the zones of high lipid concentration corresponding to the regions of where atheromatous plaques develop.

### EXAMPLE 2

### Validation of the Experimental Model

The red staining with Sudan IV made it possible to demonstrate the presence of lipidic areas corresponding to the atheromatous plaques on the aortas of the hyperlipidemic animals. These lipidic areas were absent in normolipidemic animals. The immunohistological study confirmed the expression of VCAM-1 on the atheromatous plaques of WHHL rabbits and its absence in the vessels of normolipidemic animals. Therefore the model used was adapted to the study of the binding in vivo of the VCAM-1 ligands [¹²⁵I]-B2702.84-75/75-84 and [¹²⁵I]-B2702.84-75.

### EXAMPLE 3

### Biodistribution and Blood Activity

### Euthanasia After 30 Minutes

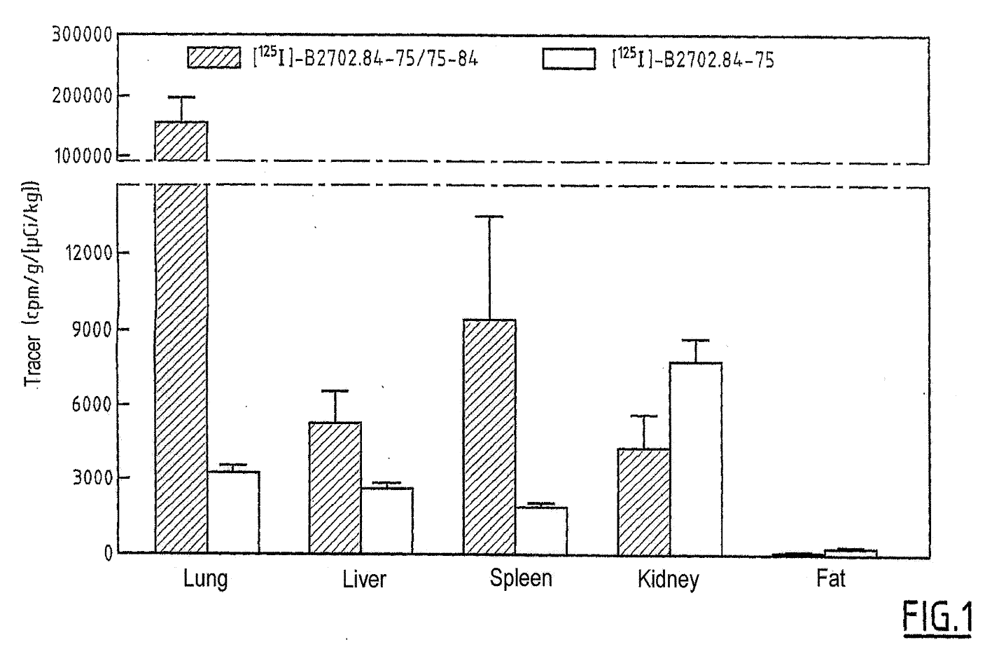

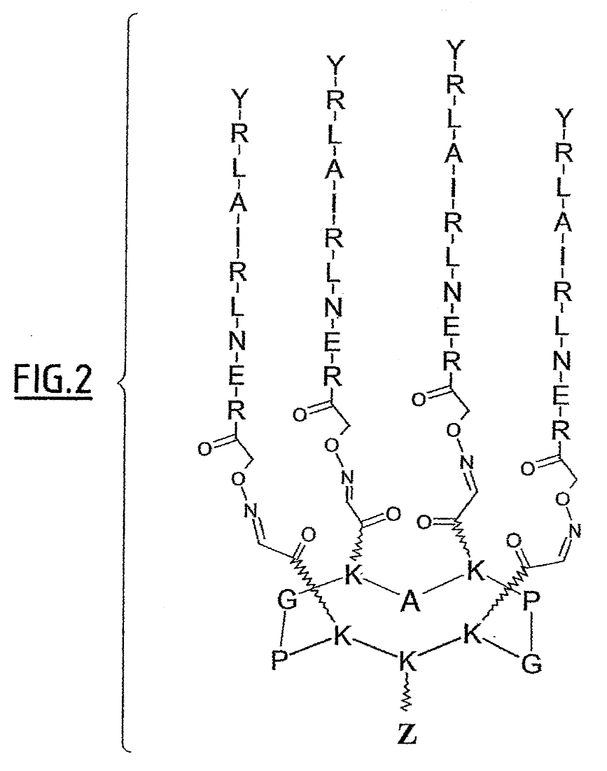

The results of the biodistribution study indicated that [¹²⁵I]-B2702.84-75/75-84 binds in a similar manner in the lung, the liver, the spleen, the kidney and the fat in normal and hyperlipidemic animals, with very strong pulmonary binding. The pulmonary binding of [¹²⁵I]-B2702.84-75 is approximately 50 times less, with an activity substantially similar to that of [¹²⁵I]-B2702.84-75/75-84 in the other organs of the WHHL rabbits (FIG. 1).

The blood activities of the two tracers reached a plateau 10 minutes after injection, which represented 40% of the initial activity. The circulatory activity observed for [¹²⁵I]-B2702.84-75 was then two times greater than that of [¹²⁵I]-B2702.84-75/75-84.

### EXAMPLE 4

### Aortic Binding

### Euthanasia after 30 Minutes

The qualitative analysis of the autoradiographic images indicates that the two tracers are preferably bound to the lipidic areas revealed by the staining with Sudan IV and which corresponded to the atheromatous plaques in WHHL rabbits. The tracer [¹²⁵I]-B2702.84-75/75-84 did not exhibit any binding on the aortas of healthy animals.

The quantification of the autoradiograhic images revealed that the binding ratio of [¹²⁵I]-B2702.84-75/75-84 between the lipidic areas and the normal aortic zones of the WHHL rabbits were 7.9±2.0, whilst the ratio of the tracer activity between the regions of stronger and weaker binding on the aortas of normal rabbits was 1.2±0.1 (p<0.01 vs WHHL group). The binding ratio of atheromatous plaque/normal aortic zone of [¹²⁵I]-B2702.84-75 in the WHHL animals was 3.0±0.3.

In conclusion, the results indicate that the two tracers bind preferentially on the atheromatous plaques expressing VCAM-1. The ratios of binding of the tracer in the region of the atheromatous plaque to binding fixation in the region of the normal aortic zone are more favourable to [¹²⁵I]-B2702.84-75/75-84. However, the tracer [¹²⁵I]-B2702.84-75 did not exhibit any notable pulmonary binding, which makes it a particularly advantageous compound for the imaging of vulnerable coronary atheromatous plaque.

### EXAMPLE 5

### Biodistribution and Blood Activity

### Euthanasia after 180 Minutes

The tracers ¹²³I-B2702.84-75 and ^{99m}Tc-B2702.84-75 were evaluated according to the same methodology as was used for Examples 3 and 4, except that the animals were euthanased 180 minutes (30 minutes previously) after the injection of the tracers.

The tracers bind in a similar manner in the organs of control rabbits and WHHL rabbits, except in the aorta where an increased tendency to bind is observed in the WHHL animals (Table 1).

TABLE 1
Biodistribution of ¹²³I-B2702.84-75 and ^{99m}Tc-B2702.84-75
180 minutes after the injection:

| | ¹²³I-B2702.84-75 | | ^{99m}Tc-B2702.84-75 | |
|---|---|---|---|---|
| | Control | WHHL | Control | WHHL |
| Tissue | (n = 3) | (n = 3) | (n = 3) | (n = 3) |
| Blood | 0.190 ± 0.038 | 0.161 ± 0.048 | 0.097 ± 0.017 | 0.102 ± 0.033 |
| Heart | 0.088 ± 0.017 | 0.088 ± 0.031 | 0.029 ± 0.008 | 0.044 ± 0.015 |
| Aorta | 0.011 ± 0.004 | 0.034 ± 0.012 | 0.025 ± 0.009 | 0.046 ± 0.012 |
| Lung | 0.138 ± 0.028 | 0.163 ± 0.044 | 0.166 ± 0.052 | 0.208 ± 0.044 |
| Liver | 0.086 ± 0.025 | 0.096 ± 0.029 | 0.427 ± 0.080 | 0.332 ± 0.042 |
| Spleen | 0.076 ± 0.015 | 0.089 ± 0.029 | 0.198 ± 0.061 | 0.172 ± 0.070 |
| Kidney | 0.307 ± 0.052 | 0.490 ± 0.184 | 0.558 ± 0.109 | 1.361 ± 0.721 |
| Fat | 0.011 ± 0.001 | 0.015 ± 0.006 | 0.012 ± 0.001 | 0.019 ± 0.009 |
| Muscle | 0.028 ± 0.003 | 0.026 ± 0.009 | 0.008 ± 0.002 | 0.017 ± 0.003 |
| Stomach | 0.095 ± 0.025 | 0.114 ± 0.040 | 0.045 ± 0.009 | 0.052 ± 0.015 |

The binding values are expressed as a percentage of the injected dose per gram of organ (% ID/g).

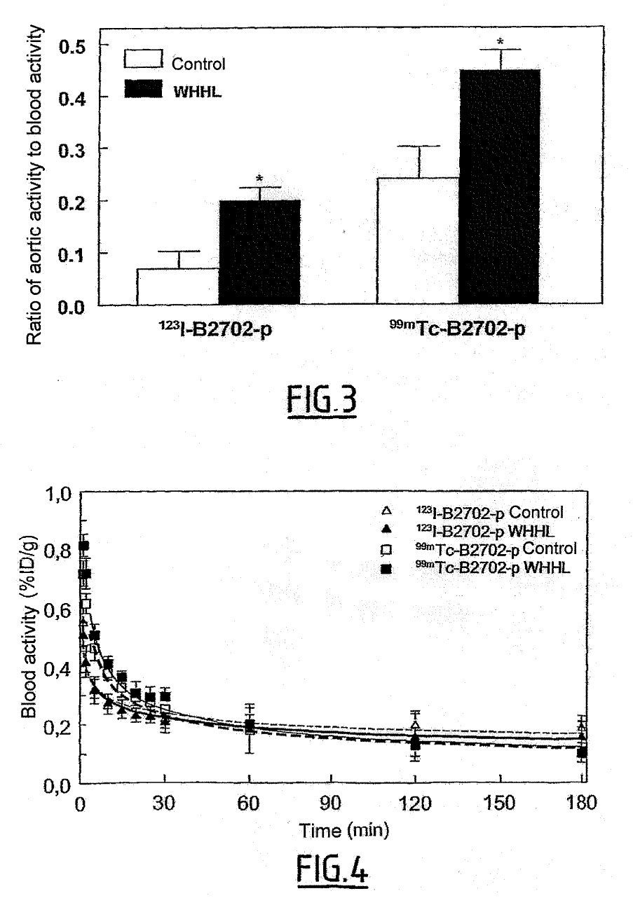

This increase becomes significant for the two tracers when the aortic activity is standardised at the circulatory tracer activity (FIG. 3).

The blood samples for measuring the circulatory tracer activity were taken 1, 2, 5, 10, 20, 25, 30, 60, 120 and 180 minutes after the injection. The blood kinetics are shown in FIG. 4. 180 minutes after the injection, the circulatory activity of ¹²³I-B2702.84-75 and ^{99m}Tc-B2702.84-75 represents respectively 30% and 15% of the initial activity. It will be recalled that the circulatory activity of ¹²⁵I-B2702.84-75 represented approximately 40% of the initial activity 30 minutes after the injection in the experiments described in Example 3.

### EXAMPLE 6

### Aortic Binding

### Euthanasia after 180 Minutes

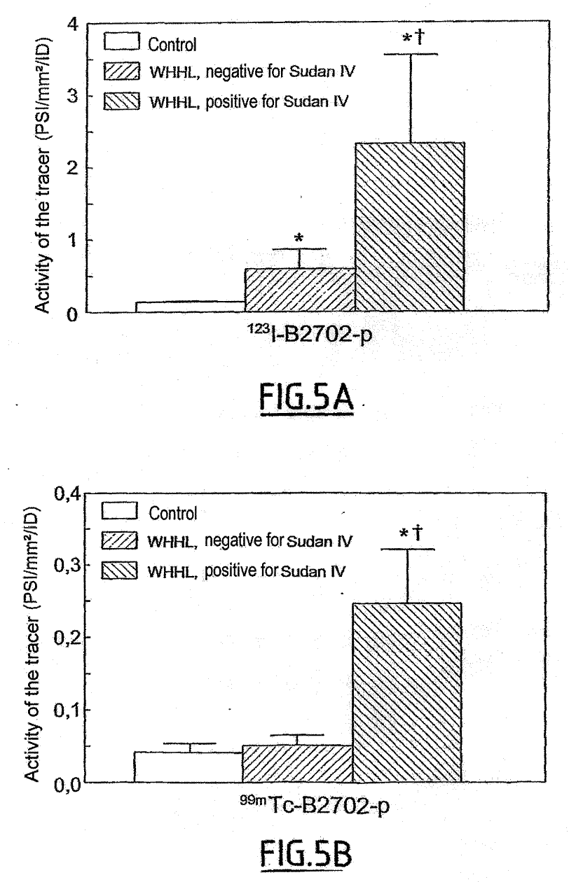

The quantification of the autoradiographic images indicates that the two tracers are preferably bound to the lipidic areas revealed by the staining with Sudan IV and corresponding to atheromatous plaques. The binding ratios of atheromatous plaque/normal aortic zone of ¹²³I-B2702.84-75 and ^{99m}Tc-B2702.84-75 in the WHHL animals are respectively 4.0 and 4.8. This ratio was 3.0 in the preceding experiments carried out 30 minutes after the injection of the tracers. Finally, the bindings of ¹²³I-B2702.84-75 and ^{99m}Tc-B2702.84-75 on the atheromatous plaques of the WHHL animals are 17.0 and 5.9 times greater than those observed on the aortas of control animals. These results are summarized in FIGS. 5A and 5B below.

### BIBLIOGRAPHY

Clubb et al. (2001) Development of atherosclerotic plaque with endothelial disruption in Watanabe Heritable Hyperlipidemic rabbit aortas. Cardiovasc Pathol. 9: 1-11
Colas P, Cohen B, Jessen T, Grishina I, McCoy J, Brent R. (1996) Genetic selection of peptide aptamers that recognize and inhibit cyclin-dependent kinase 2. Nature, 380, 548-50.
Checovich W. J., Bolger R. E., Burke T. (1995) Fluorescence polarization—a new tool for cell and molecular biology. Nature; 375(6528):254-6.
Fayad Z A and Fuster V (2001) Clinical imaging of the high-risk or vulnerable atherosclerotic plaque. Circ Res.; 89:305-316
Jayasena S. D. (1999) Aptamers: an emerging class of molecules that rival antibodies in diagnostics. Clin Chem. 45(9):1628-50.
Ling X et al. (2000) An immunosuppressive and anti-inflammatory HLA class I-derived peptide binds vascular cell adhesion molecule-1. Transplantation. 70 :662-667 Narula J et al. (1999) Strategic targeting of atherosclerotic lesions. J Nucl Cardiol.
Osborn L et al. (1989) Direct expression cloning of vascular cell adhesion molecule 1, a cytokine-induced endothelial protein that binds to lymphocytes. Cell. (6):1203-11
Tuerk C. and Gold L. (1990) Systematic evolution of ligands by exponential enrichment: RNA ligands to bacteriophage T4 DNA polymerase. Science. 3; 249(4968):505-10.

1. A method of non-invasive medical imaging in vivo which comprises a step of detection of a contrast agent, which contrast agent is a VCAM-1 ligand.

2. The method according to claim 1, in which the medical imaging is a technique selected from within the group consisting of scintigraphy, MRI and optical imaging.

3. The method according to claim 1, in which the VCAM-1 ligand is selected from within the group consisting of B 2702-84.75/75-84, B 2702.84-75, 4(B 2702.84-75)Raft, and VCAM-1 ligand aptamers.

4. The method according to claim 1, in which the VCAM-1 ligand is associated with a marker.

5. The method according to claim 4, in which the said marker is selected from within the group consisting of a radioactive nuclide, a fluorophore, a chemiluminescent compound, a bioluminescent compound and a MRI contrast agent.

6. The method according to claim 4, in which the said marker is selected from within the group consisting of ¹²³I, ¹²⁵I, ^{99m}Tc, ¹¹¹In, ¹⁸F, ⁶⁷Ga, nanoparticles, gadolinium, fluorescein, Alexa, cyanine, luciferase and alkaline phosphatase.

7. The method according to of claim 1, in which the VCAM-1 ligand is labelled and selected from within the group consisting of [¹²⁵I]-B 2702.84-75/75-84, [¹²⁵I]-B 2702.84-75, [¹²³I]-B 2702.84-75/75-84, [¹²³I]-B 2702.84-75, [^{99m}Tc]-B 2702.84-75/75-84, [^{99m}Tc]-B 2702.84-75, [¹²³I]-4(B 2702.84-75)Raft, [¹²⁵I]-4(B 2702.84-75)Raft, [^{99m}Tc]-4(B 2702.84-75)Raft, and VCAM ligand aptamers labelled with iodine 123, iodine 125 or Tc99m.

8. The method according to claim 1, for the detection of a cardiovascular disease and/or a risk of an acute ischaemic event in a subject likely to exhibit such a disease.

9. The method according to claim 8, in which an atheromatous plaque is detected.

10. The method according to claim 9, in which the atheromatous plaque is a vulnerable atheromatous plaque.

11. The method according to claim 9 or in which the atheromatous plaque is a vulnerable coronary atheromatous plaque.

12. The method according to claim 8, in which the said cardiovascular disease is a coronary disease.

13. The method according to claim 8, in which the said risk of an acute ischaemic event is a risk of myocardial infarction.

14. The method according to claim 8, in which the atheromatous plaque is a carotid or renal atheromatous plaque, or is located in the region of an artery of the lower limbs.

15. The method according to claim 8, in which the said risk of acute ischaemic event is a risk of renal embolism.

16. The method according to claim 8, in which the said risk of acute ischaemic event is a risk of cerebral vascular accident.

17. The method according to claim 1, for the therapeutic monitoring of a cardiovascular disease in a subject in which a cardiovascular disease has been diagnosed.

18. A method for treating or preventing a cardiovascular disease in an individual, wherein a therapeutically prophylactically effective amount of a VCAM-1 ligand is administered to said individual.

19. The method according to claim 18, in which the said VCAM-1 ligand is coupled with a cytotoxic agent.

20. The method according to claim 19, in which the said cytotoxic agent is a radioactive isotope selected from within the group consisting of iodine 131 and yttrium 90 (⁹⁰Yt).

21. The method according to claim 18, in which the said VCAM-1 ligand is selected from within the group consisting of the peptide B 2702.84-75/75-84, the peptide B 2702.84-75, the compound 4(B 2702.84-75)Raft, and VCAM ligand aptamers.

22. The method according to claim 18, in which the said VCAM-1 ligand is used for treating a vulnerable coronary or aortic atheromatous plaque.

23. The method according to claim 18, in which the said VCAM-1 ligand is used for the prevention of an acute ischaemic event selected from within the group consisting of a myocardial infarction, a cerebral vascular accident, a renal embolism, acute ischaemia of a limb, and a rupture of an aortic aneurism.


SEQUENCE LISTING (4 sequences)

SEQ ID NO: 1 - protein, 10 aa, Homo sapiens
YRLAIRLNER

SEQ ID NO: 2 - protein, 20 aa, Homo sapiens
YRLAIRLNERRENLRIALRY

SEQ ID NO: 3 - protein, 12 aa, Homo sapiens
YRLAIRLNERGH

SEQ ID NO: 4 - protein, 10 aa, Homo sapiens
GKAKPGKKKP